(19) United States
(12) Patent Application Publication
(10) Pub. No.: US 2011/0172721 A1
(43) Pub. Date: Jul. 14, 2011

(54) ORTHOPEDIC IMPLANTS FOR USE WITH PRECISION BONE RESURFACING INSTRUMENTATION

(75) Inventors: Robert Frigg, Bettlach (CH); Samuel Leuenberger, Oberwil (CH); Jens Richter, Basel (CH); Christopher Stabley, West Chester, PA (US)

(73) Assignee: Synthes (U.S.A.), West Chester, PA (US)

(21) Appl. No.: 12/666,304

(22) PCT Filed: Jun. 27, 2008
(86) PCT No.: PCT/US08/68606

§ 371 Date: Nov. 11, 2010

Related U.S. Application Data
(60) Provisional application No. 60/947,254, filed on Jun. 29, 2007.

(51) Int. Cl. A61B 17/86

(52) U.S. Cl. 606/329

(57) ABSTRACT

Reduced height and zero-profile implants, such as bone plates, are provided that take advantage of improved bone resurfacing instrument technology to form precisely resurfaced implant-receiving beds in cortical bone, such as across a fracture or bone portion in need of repair, to accommodate the implants. The implants are provided in forms that do not necessitate the inclusion of bone screws while providing increased implant-repulsion resistance. The implants may be sized and configured to receive anchoring pins, preferably anchoring pins having non-circular cross-sectional shafts that further increase implant-repulsion resistance.

The present application claims priority to U.S. provisional application 60/947,254, filed Jun. 29, 2007.

### FIELD OF TECHNOLOGY

The present invention relates generally to orthopedics. More specifically, the present invention relates to implants for supporting and allowing the repair and regeneration of skeletal members in need thereof.

### BACKGROUND

It is known in the art to implant a bone plate atop a bone surface and across a fracture site or other skeletal defect in need of repair. It is also known in the art to secure (e.g., anchor) the bone plate to the underlying bone with bone screws. Bone plates however are prone to repulsion, due to the stresses imparted onto the bone plates and bone screws. Such implant failures in orthopedics is undesirable. That is, one of the most common occurrences of implant failure in orthopedics occurs when the bone screws back-out from the bone plates, a complication that may lead to serious consequences in a patient. Due to the general cylindrical shape of bone screws and the variety of forces acting thereon, during and after the healing of a bone, once a bone screw begins to dislodge from the bone or otherwise lose purchase, there is little to prevent a loosened bone screw from continuing to back out from the bone and the bone plate, potentially puncturing surrounding tissue, such as, has been the case in cervical anterior plate failures in which patients reportedly swallow or even cough up expulsed anterior cervical plate screws that puncture the esophageal lining. The bone plate from which such a bone screw has become dislodged becomes even less securely implanted and the chances of additional screws backing out and complete implant failure increases dramatically.

Implants, such as bone plates and bone screws, are less likely to fail due to screw back-out or implant repulsion when such implants are designed with irregular, i.e., non-rounded, shapes. In addition, implants, such as bone plates implanted partially or entirely under the surface of the bone are much less likely to fail due to repulsion. However, bone resurfacing technology, up until now, has not enabled implants such as bone plates to be inserted under the surface of the bone in part due to the difficulty with conventional mechanical bone resurfacing instrumentation, such as millers, rasps, and drills, in forming sharp edges or precisely resurfaced areas. Moreover, handling such instrumentation in the confined surgical areas is difficult and invasive for surgeons. Other difficulties include the possibility of breaching the vascularized bone underlying the cortical shell during the use of such instrumentation to mill or otherwise resurface a topical bone area, the risk of the resurfacing instrument slipping off of the slippery bone surface and causing damage to surrounding tissue or vessels, and the risk of greatly reducing the strength or integrity of the bone tissue immediately surrounding the machined bone surface. For example, it has conventionally been extremely difficult to form slots or grooves in a patient's bone using mechanical instruments, such as saws, due to the tendency for the saw to damage and/or destroy adjacent soft tissue in the process. These problems are further exacerbated when attempting to form a slot, groove or implant-receiving bed in the skull bone due to the relative thinness of the skull bone as well as the delicate tissue structure underlying the skull bone.

Recently, with the advent of improved bone resurfacing technologies, such as, for example, lasers, radio frequency RF and other electromagnetic bone resurfacing instruments, piezo-activated resurfacing instruments, piezoelectric cutting knives, water jets, and other precision bone milling instrumentation, etc. comes the opportunity to insert implants, such as bone plates, into partially and/or wholly implant-receiving slots, grooves, or beds formed in the surface of the bone in need of repair. Pulsed lasers, for example, have been developed that are capable of sending sensing signals between energy pulses that enable the laser to cut, for example, through the outer shell of a hard-boiled egg yet not damage the delicate membrane underlying the shell.

Such implantation would have reduced probability of implant repulsion. Additional advantages of improved bone resurfacing technologies include providing implants having non-rounded edges and/or non-threaded bone anchors for use with such implants that are characterized as having noncircular cross-sectional anchor shafts.

A need exists to take advantage of improved bone resurfacing technology to provide orthopedic implants having reduced profiles and enhanced repulsion-resistance characteristics.

### SUMMARY

Reduced height and zero-profile implants, such as bone plates, are provided. The implants are adapted for placement across a skeletal defect, such as a fracture, in need of repair. Further, the implants are provided in forms that provide increased implant-repulsion resistance.

In one embodiment, the implant may be in the form of a biocompatible wire. In a preferred embodiment, the wire is inserted into a curvilinear groove formed using a bone milling or resurfacing instrumentation such as, for example, a laser, radio frequency RF resurfacing instrument, other electromagnetic or mechanical resurfacing instrument. The curvilinear groove into which the wire is implanted preferably crosses the fracture site, such that upon implantation of the wire into the groove, the wire acts to secure the two bone fragments.

In accordance with one aspect of the invention, the wire may have a trapezoidal transverse cross section, where the distally implanted surface of the wire has a width that is larger than the proximally implanted surface, and may be implanted into a groove that has a substantially similar cross-sectional shape. In this manner, expulsion of the wire is less likely. Additionally, the wire may be formed with a material that is expandable once introduced into the patient's body.

In accordance with another aspect of the invention, the implanted wire and/or the surgically-formed groove may be covered with a biocompatible adhesive to anchor the implant with respect to the surrounding bone tissue. The adhesives may be inserted into the groove and/or around or on top of the wire implant either prior to, during, or subsequent to the implantation of the implant wire. Alternatively, the wire may be affixed to the bone with bone anchors.

In a preferred embodiment, the wire may have a diameter that is about one millimeter, and the groove into which the wire is implanted may have a similar depth of one millimeter, such that the proximal surface of the wire lies substantially flush (e.g. even) with or below the top surface of the bone and the depth of the groove does not extend below the cortical bone.

In another embodiment, the wire implant may also include extensions, such as, for example, barbs, filaments, or clips along the shaft of the wire. The extensions may be integrally formed with the wire implant. Alternatively, the extensions may be formed independently of the wire and attached thereto. Additionally, the wire implant may be configured with arrowheads at opposite ends of its length. In this manner, the arrowheads may assist in compressing the two bone fragments across the fracture site while securing the wire implant in place within the groove. Accordingly, the machining and/or lasering of the implant-accommodating groove may include surface cutting of one or more areas adjacent to the curvilinear groove to facilitate insertion of the implant wire and to accommodate the additional securing means.

In another embodiment, the implant may be in the form of a bone plate. In a preferred embodiment, the bone plate assumes a form having two enlarged end portions with an intermediary connecting or bridge portion located therebetween, wherein each enlarged end portion may include an optional bore hole for optional screw fixation. In use, the bone plate may be applied across a fracture site or other bone region in need of repair.

In accordance with another aspect of the invention, a plate-receiving recess is formed in the bone using a bone milling or resurfacing instrumentation. The bone plate is preferably inserted at least partially or wholly into the machined plate-receiving recess. The bone plate serves to hold the bone pieces across the fracture site securely with respect to one another to assist in fusion. Preferably, the thickness of the bone plate substantially corresponds to the depth of the machined plate-receiving recess or otherwise resurfaced area of bone such that, once implanted, the top surface of the bone plate lies substantially flush with or below the top surface of the bone, thus a zero-height implant is provided.

In accordance with one aspect of the invention, the plate may have a trapezoidal transverse cross section, where the distally implanted surface of the wire has a width that is larger than the proximally implanted surface, and may be implanted into a recess that has a substantially similar cross-sectional shape. In this manner, expulsion of the plate is less likely. Additionally, the plate may be formed with a material that is expandable once introduced into the patient's body.

In accordance with another aspect of the invention, the implanted plate and/or the recess may be covered with a biocompatible adhesive to anchor the implant with respect to the surrounding bone tissue. The adhesives may be inserted into the recess and/or around or on top of the plate either prior to, during, or subsequent to the implantation of the implant wire. Alternatively, the plate may be affixed to the bone using bone anchors.

### BRIEF DESCRIPTION OF THE DRAWINGS

The system is explained in even greater detail in the following exemplary drawings. The drawings are merely exemplary to illustrate the structure of preferred devices and certain features that may be used singularly or in combination with other features. The invention should not be limited to the embodiments shown.

FIG. 1 illustrates a wire-like implant and an associated orthopedic fixation method in accordance with one aspect of the present invention;

FIG. 2 illustrates a wire-like implant having barb-like members and an associated orthopedic fixation method in accordance with another aspect of the present invention;

FIG. 3 illustrates a wire-like implant having alternate anchoring structures and an associated orthopedic fixation method in accordance with another aspect of the present invention;

FIG. 4 illustrates a wire-like implant having alternate anchoring structures and an associated orthopedic fixation method in accordance with another aspect of the present invention;

FIG. 5 illustrates a zero profile bone plate and associated implantation method in accordance with another aspect of the present invention;

FIG. 6 illustrates a zero profile bone plate and associated implantation method in accordance with another aspect of the present invention;

FIG. 7 illustrates a variety of zero profile bone plate designs that may be used, for example, in cranio- and maxillofacial reconstruction, in accordance with another aspect of the present invention;

FIG. 8 illustrates a human skull with skeletal reconstruction plates in accordance with another aspect of the present invention;

FIG. 9 illustrates a variety of plates with respect to bone cross sectional profiles in accordance with another aspect of the present invention;

FIG. 10 illustrates additional bone plate and bone anchor systems in accordance with another aspect of the present invention;

FIG. 11 illustrates a spring-biased cruciform spring clip for orthopedic fixation in accordance with another aspect of the present invention;

FIG. 12 illustrates a wave blade for orthopedic fixation in accordance with another aspect of the present invention;

FIG. 13 illustrates a wave blade for orthopedic fixation in accordance with another aspect of the present invention;

FIG. 14 illustrates a staple-type implant having legs with square cross-sectional areas in accordance with another aspect of the present invention;

FIG. 15 illustrates another embodiment of the staple-type implant in accordance with another aspect of the present invention;

FIG. 16 illustrates a skeletal fixation implant having a taper along its depth for reduced implant repulsion probability and an associated implantation method in accordance with another aspect of the present invention;

FIG. 17 illustrates a variety of alternate implant designs in accordance with another aspect of the present invention;

FIG. 18 illustrates a non-straight (e.g. snake-like or crooked) bone cut, which can be filled by an injectable material in accordance with another aspect of the present invention;

FIG. 19 illustrates a non-straight (e.g. snake-like or crooked) bone cut, which can be filled with a soft and/or malleable, but non-liquid material in accordance with another aspect of the present invention;

FIG. 20 illustrates a stencil or template-type instrument to guide the bone cutting tool; and

FIG. 21 illustrates the use of a numerical controlled guiding system for controlled bone removal.

### DETAILED DESCRIPTION OF ILLUSTRATIVE EMBODIMENTS

Certain exemplary embodiments of the invention will now be described with reference to the drawings. In general, such embodiments relate to a skeleton fixation system 10 for securing bones across a fracture site. As generally understood by one of ordinary skill in the art, it should be understood that while the skeleton fixation system 10 may be described in connection with cranio or maxillofacial fixation, those skilled in the art will appreciate that the system as well as the components thereof may be used for fixation in other parts of the body such as, for example, in the long bones or bones in the hand, face, feet, etc.

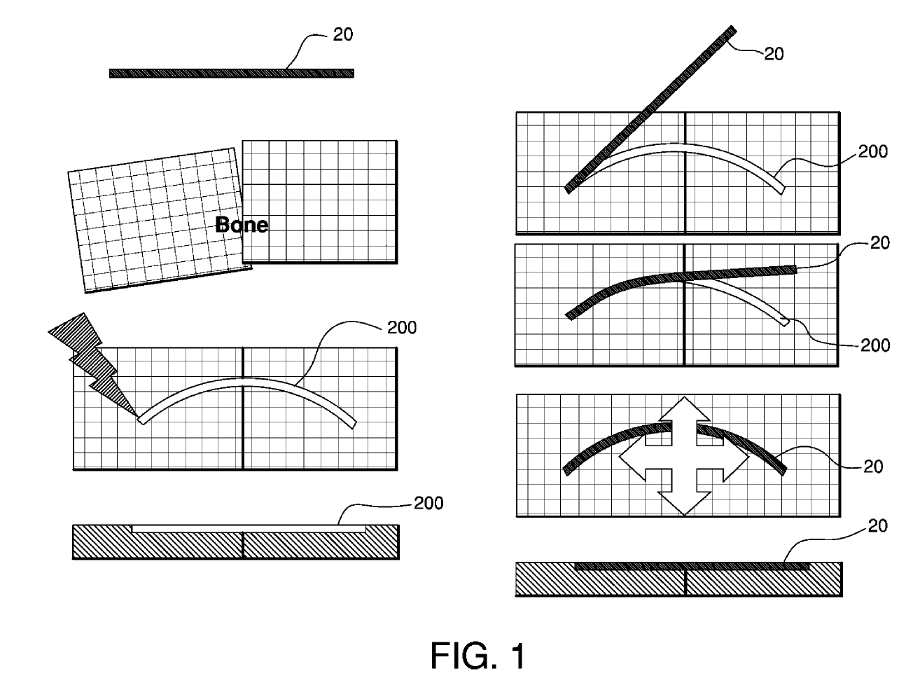

As shown in FIG. 1, a skeletal fixation member for placement across a skeletal defect, such as a fracture, in need of repair may be in the form of a biocompatible wire 20. The wire 20 preferably being at least partially embedded into the cortical bone across the skeletal defect to secure the skeletal area and/or to enable fusion. In a preferred embodiment, the wire 20 is inserted into a curvilinear groove 200 formed using a bone milling or resurfacing instrumentation such as, for example, a laser, radio frequency RF resurfacing instrument, other electromagnetic or mechanical resurfacing instrument. The curvilinear groove 200 into which the wire 20 is implanted preferably crosses the fracture site at or near the apex of its arc, such that upon implantation of the wire 20 into the groove 200, the wire 20 acts to secure the two bone fragments and resists forces acting parallel to the top bone surface.

The wire 20 may have any cross-sectional shape and/or area known in the art including but not limited to cylindrical, rectilinear, trapezoidal, polygonal, etc. Where the wire 20 has a trapezoidal shape, the distally implanted surface of the wire 20 may have a width that is larger than the proximally implanted surface, and may be implanted into a groove 200 that has a substantially similar cross-sectional shape and/or dimensions. In this manner, expulsion of the wire 20 is less likely. The wire 20 may be implanted by, for example, lacing the wire 20 through one end, inserting the implant down from above with some force, snapping the implant into the receiving bed, distracting the bone segments so that the groove 200 is slightly enlarged as may be practical in the case where there is a complete fracture, etc.

In addition, by selecting the appropriate choice of material, the wire 20 may further be expandable once introduced into the patient's body or bone tissue. Alternatively and/or in addition, the wire 20 maybe drug-eluting and/or coated with a tissue-ingrowth-enhancing material, such as, for example, BGH or hydroxyapatite.

Alternatively and/or in addition, the implanted wire 20 and/or the surgically-formed groove 200 may be covered with a biocompatible adhesive such as, for example, bone putty, cyanoacrylates, polyurethanes, epoxies, acrylics, calcium phosphate cement, etc. to provide a more secure anchoring of the implant with respect to the surrounding bone tissue. It is envisioned that the adhesives may be inserted into the groove 200 and/or around or on top of the wire 20 implant either prior to, during, or subsequent to the implantation of the implant wire 20.

The wire 20 may be formed of any biocompatible material known in the art meeting the strength and flexibility requirements of the particular applications including but not limited to stainless steel, titanium, Ni—Ti (nitinol), Elgiloy, other shape memory alloys, polymers such as PEEK, bioresorbable materials, etc.

In a preferred embodiment, the wire 20 may have a diameter that is about one millimeter, and the groove 200 into which the wire 20 is implanted may have a similar depth of one millimeter, such that the proximal surface 120 of the wire 20 lies substantially flush (e.g. even) with or below the top surface of the bone and the depth of the groove 200 does not extend below the cortical bone.

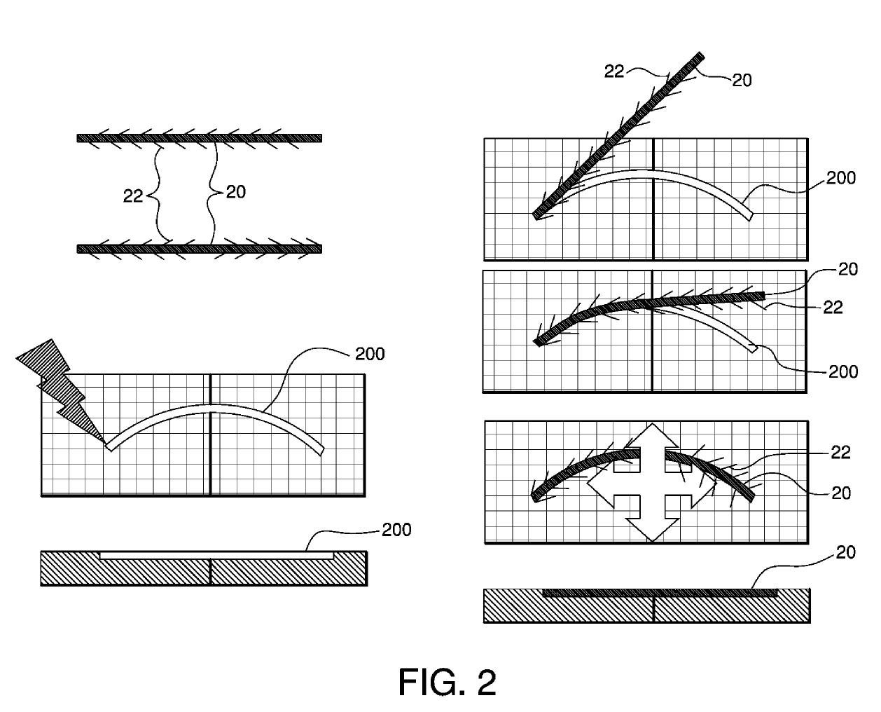

As shown in FIG. 2, the wire 20 implant may also include extensions 22 such as, for example, barbs, filaments, or clips along the shaft of the wire 20. The extensions 22 may provide additional purchase into the surrounding cortical bone tissue and provide a more secure anchoring of the wire 20 implant with respect to the surrounding tissue. The extensions 22 may be integrally formed with the wire 20 implant. Alternatively, the extensions 22 may be formed independently of the wire 20 and attached thereto. As such, the extensions 22 may be formed from the same material as the wire 20 implant, or they may be formed from a different material, such as, for example, of nitinol, Elgiloy, etc. The extensions 22 may also be postoperatively or intraoperatively deployable. That is, for example, the extensions 22 may be mechanically or magnetically deployable. Alternatively, the extensions 22 may be permanently arranged on the exterior surface of the wire 20.

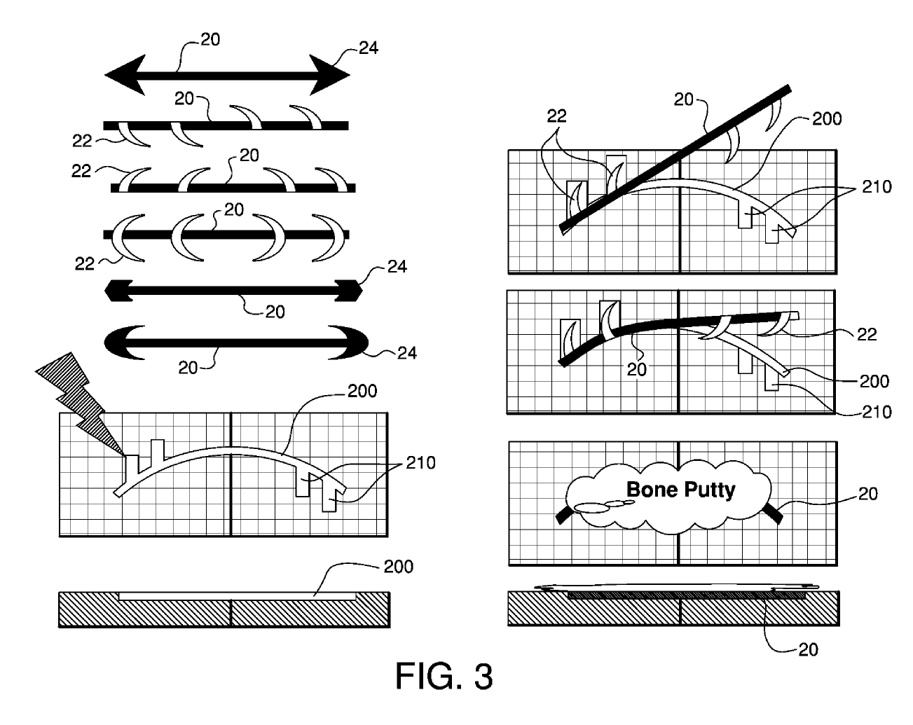

As shown in FIG. 3, various additional securing means are contemplated to assist in anchoring the wire 20 implant with respect to the surrounding bone tissue. In one example, the wire 20 implant may be configured with arrowheads 24 at opposite ends of its length. In this manner, the arrowheads 24 may assist in compressing the two bone fragments across the fracture site while securing the wire 20 implant in place within the groove 200. FIG. 3 also shows various additional configurations for providing enhanced securement of the wire 20 implant with respect to bone tissue.

As shown, the machining and/or lasering of the implant-accommodating groove 200 may include surface-cutting of one or more areas 210 adjacent to the curvilinear groove 200 to facilitate insertion of the implant wire 20 to accommodate the variously depicted additional securing means.

Alternatively and/or in addition, as previously stated, the implanted wire 20 and/or the surgically-formed groove 200 may be covered with a biocompatible adhesive such as, for example, bone putty, cyanoacrylates, polyurethanes, epoxies, acrylics, calcium phosphate cement, etc. to provide a more secure anchoring of the implant with respect to the surrounding bone tissue. It is envisioned that the adhesives may be inserted into the groove 200 and/or around or on top of the wire 20 implant either prior to, during, or subsequent to the implantation of the implant wire 20.

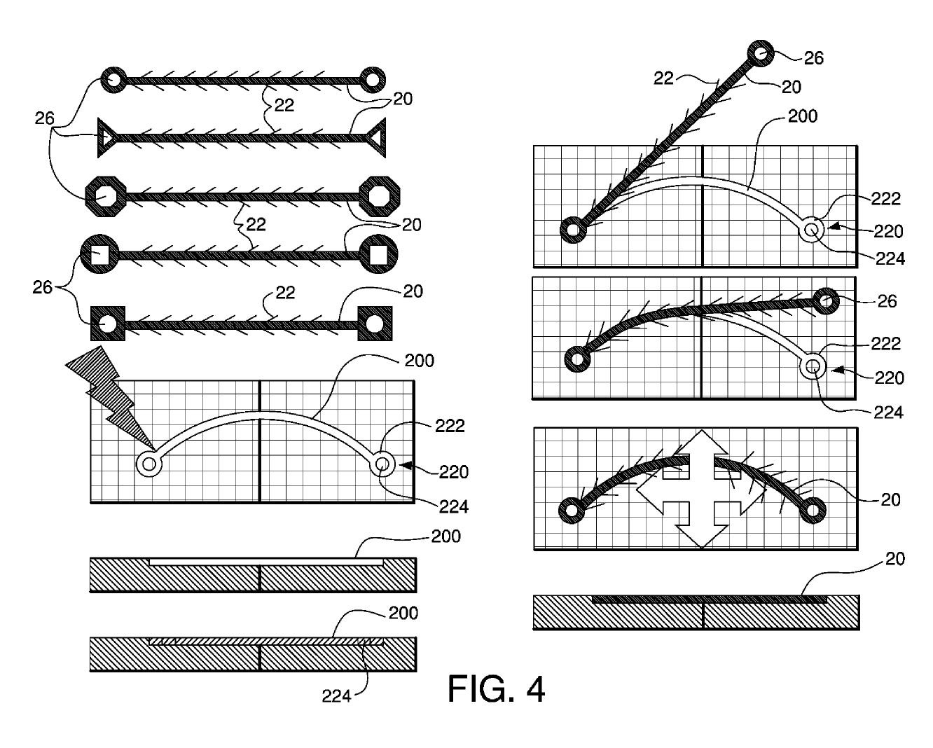

Alternatively and/or in addition, as shown in FIG. 4, the groove 200 may also include one or more circular machined areas 220. As shown, the circular machined areas 220 maybe located at opposite ends of the curvilinear grooves, the circular areas including a hollow circular recess 222, the recess preferably having a depth similar to that of the curvilinear groove 200. The hollow circular recesses preferably surrounds one or more cylindrical bone peg 224 that are formed by not machining or lasering, such that the cylindrical bone pegs 224 lie flush with the top bone surface that are unmachined or unlaser-treated. The curvilinear groove 200 and hollow circular recesses are preferably sized and configured to receive a wire 20 implant having hollow rings or eyelets 26 disposed at opposite ends thereof so that the eyelets 26 surround the cylindrical bone pegs 224 left during the machining or lasering of the groove 200 thus facilitating a secure implantation with additional repulsion-resistance.

As shown, the eyelets 26 and corresponding bone pegs 224 may assume a circular form. Alternatively, the eyelets 26 and corresponding bone pegs 224 may assume a non-circular form such as, for example, a square or polygonal shape, which due to their sharp edges provided additional resistant to repulsion as compared to circular forms. Alternatively and/or in addition, the bone pegs 224 may assume a noncircular form, such as, for example, a square or polygonal shape for mating with a circular ring having a corresponding square or polygonal eyelet hole 26. Alternatively, the bone pegs 224 may assume a cylindrical form while the ring member may have a square or polygonal exterior surface with a circular eyelet hole 26.

As will be appreciated by one of ordinary skill in the art the machined areas and corresponding eyelets 26 may be formed anywhere along the length of the wire 20 and/or groove 200.

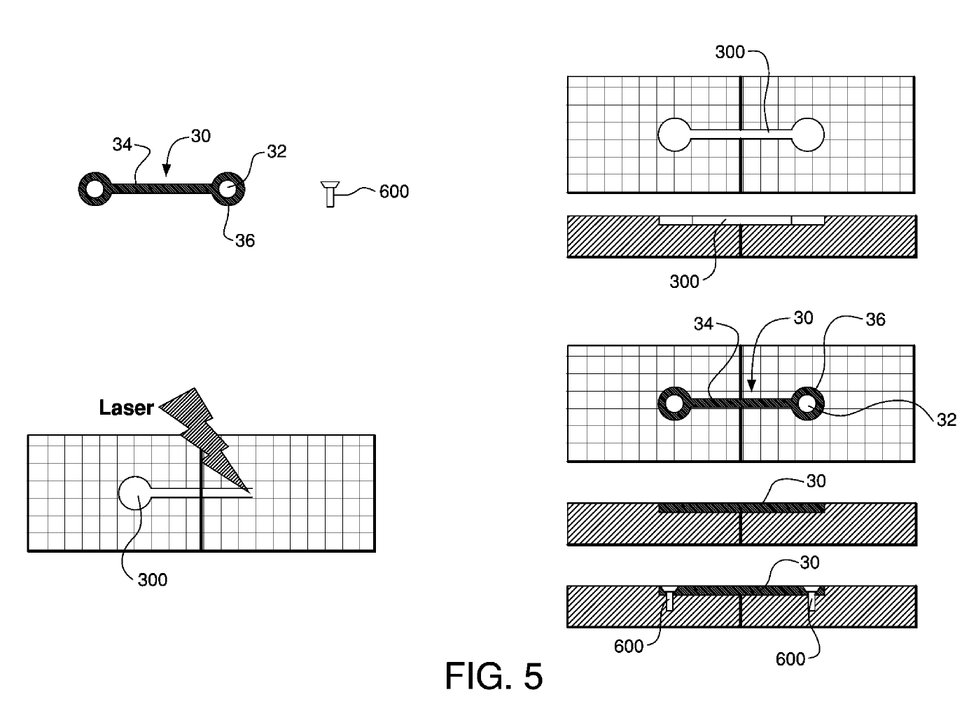

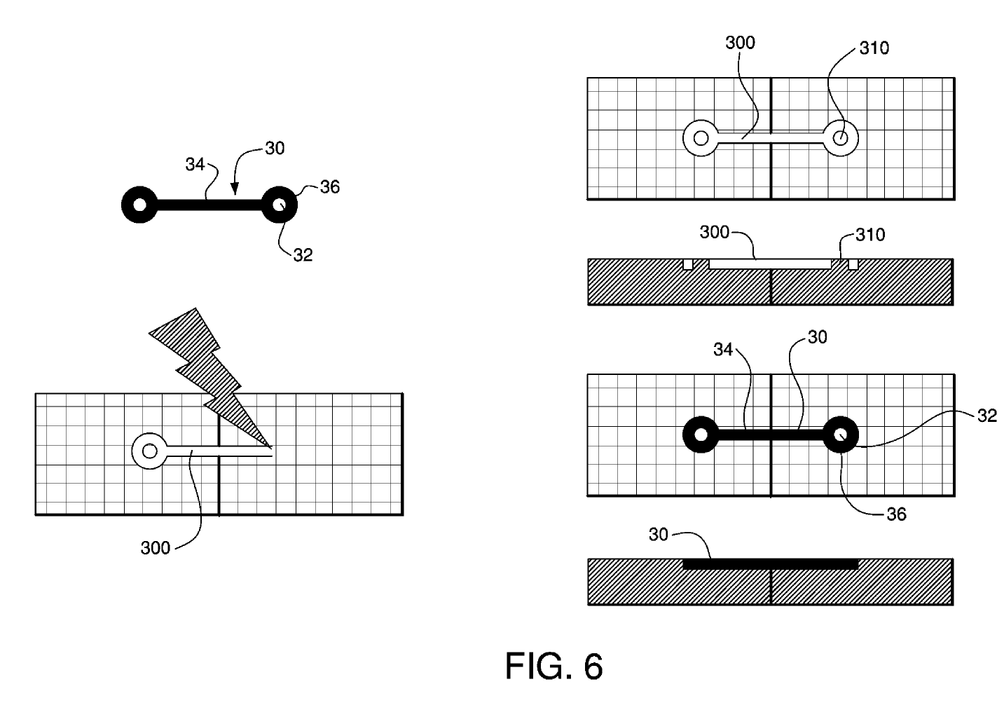

As shown in FIGS. 5 and 6, a particularly well suited cranio or maxillofacial bone plate 30 is depicted. Although as will be appreciated by one of ordinary skill in the art, the bone plate 30 may be used in other parts of the body as well. The cranio or maxillofacial bone plate 30 is similar in design and geometry to conventional cranio or maxillofacial bone plates in that the bone plate 30 preferably has a thin profile including small-diameter screw-receiving bore holes 32 connected with a thin intermediary plate area 34.

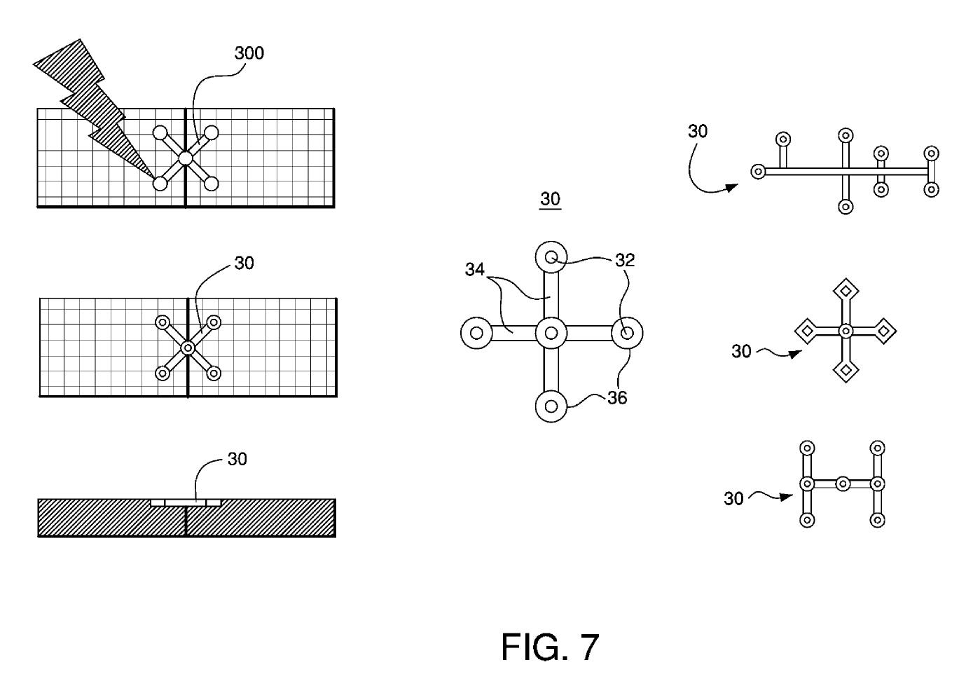

That is, as shown, the cranio- or maxillofacial bone plate 30 preferably assumes a form having two enlarged end portions 36 with an intermediary connecting or bridge portion 34 located therebetween, wherein each enlarged end portion 36 may include an optional bore hole 32 for optional screw fixation. As such, the cranio or maxillofacial bone plate 30 may assume the general form of a barbell that includes two enlarged rounded lobes 36 at either end connected by an intermediate linking portion 34 having a dimension smaller in width than either of the lobes 36. Each of the lobes 36 may include a bore hole 32 for optional screw fixation. The bore holes 32 may further be configured to fit over bone pegs similar to those discussed above with reference to FIG. 5 instead of accommodating bone screws as seen in FIG. 7. Alternatively, the lobes 36 may be free or devoid of any boreholes 32.

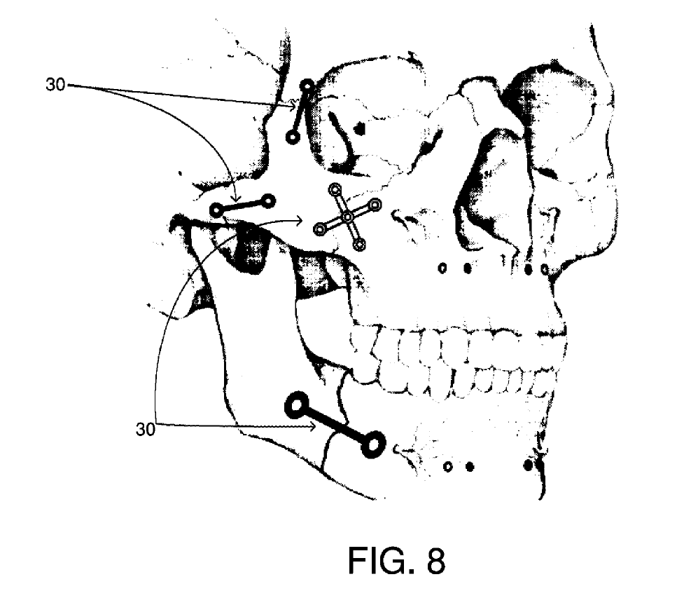

In use, as shown in FIG. 8, the bone plate 30 may be applied across a fracture site or other bone region in need of repair. A plate-receiving area 300 is formed in the bone using a bone milling or resurfacing instrumentation such as, for example, a pulsed laser, a radio frequency RF resurfacing instrument, a mechanical resurfacing instrument, etc. The bone plate 30 is preferably inserted at least partially or wholly into the machined plate-receiving area 300. The bone plate 30 serves to hold the bone pieces across the fracture site securely with respect to one another to assist in fusion. Preferably, as previously stated, the thickness of the bone plate 30 substantially corresponds to the depth of the machined plate-receiving area 300 or otherwise resurfaced area of bone such that, once implanted, the top surface of the bone plate 30 lies substantially flush with or below the top surface of the bone, thus a zero-height implant is provided.

Moreover, as previously stated, the bone plate 30 implant may be expandable once introduced into the patient's body or bone tissue with the appropriate choice of material. The bone plate implant may also be drug-eluting and/or coated with a tissue-ingrowth-enhancing material, such as, for example, BGH or hydroxyapatite. The surface of the bone plate implant may also include texturing to assist with bone in-growth. Alternatively and/or in addition, the implanted bone plate 30 and/or the machined plate-receiving area 300 may be covered with a biocompatible adhesive. The bone plate 30 and/or the bone screws may further be bioresorbable.

As shown in FIG. 7, various alternate sized and shaped bone plates 30 are depicted. Once again, these plates 30 are particularly well suited for cranio- or maxillofacial applications but as will be appreciated by one of ordinary skill in the art, the bone plates 30 may be used in other parts of the body as well. As shown, the bone plates 30 may have generally more complex plate designs, which are particularly suited for more complex fracture reduction and/or fusion. In one preferred embodiment, the zero-profile cruciform shaped plate 30 has a general X-shape in which connecting members 34 join boreholes 32 at opposite ends 36 of each connecting member 34 for receiving bone anchors or bone pegs 310. The cruciform plate 30 may be oriented and/or inserted into a corresponding machined or lasered groove 300 spanning a fracture site such that two anchoring means are positioned on either side of the fracture. Similarly, the more complex plate designs illustrated in FIG. 7 may be implanted into corresponding grooves 300 formed into the bone surfaces such that a plurality of anchoring means are situated on one or more sides of a bone fracture.

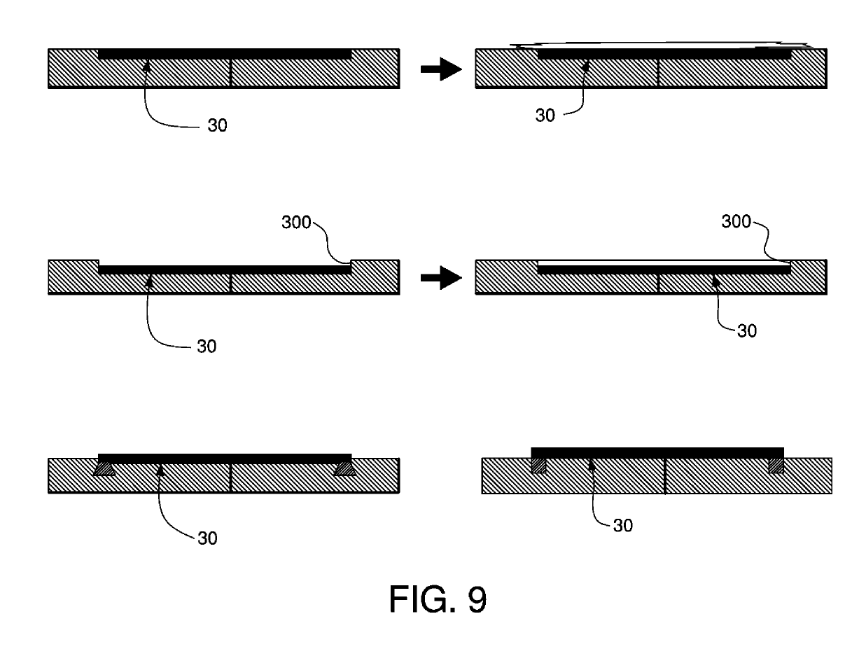

As shown in FIG. 9, according to one aspect of the present invention, the implants (e.g., wire 20, plates, etc.) may be implanted into a corresponding machined or lasered area 300 of bone such that the top surface of the implant lies substantially flush with or below the top surface of the bone. Alternatively, the top surface of the implant may lie slightly below (e.g. recessed) with respect to the top surface of the bone or slightly above the top surface of the bone. Alternatively and/or in addition, a biocompatible adhesive such as, for example, a bone putty, can be applied atop the implant to further assist in fusing the two bone segments in need of repair. As previously stated, the bone plate 30 preferably has a thickness designed for the particular application and can be in the range of about 0.5 to about 5 millimeters (in some cases, for example, approximately one millimeter in thickness is useful) and is received in an implant-receiving bed that is surgically formed into the top surface of the bone to a depth of equal or slightly greater depth (in some cases, for example, about one millimeter).

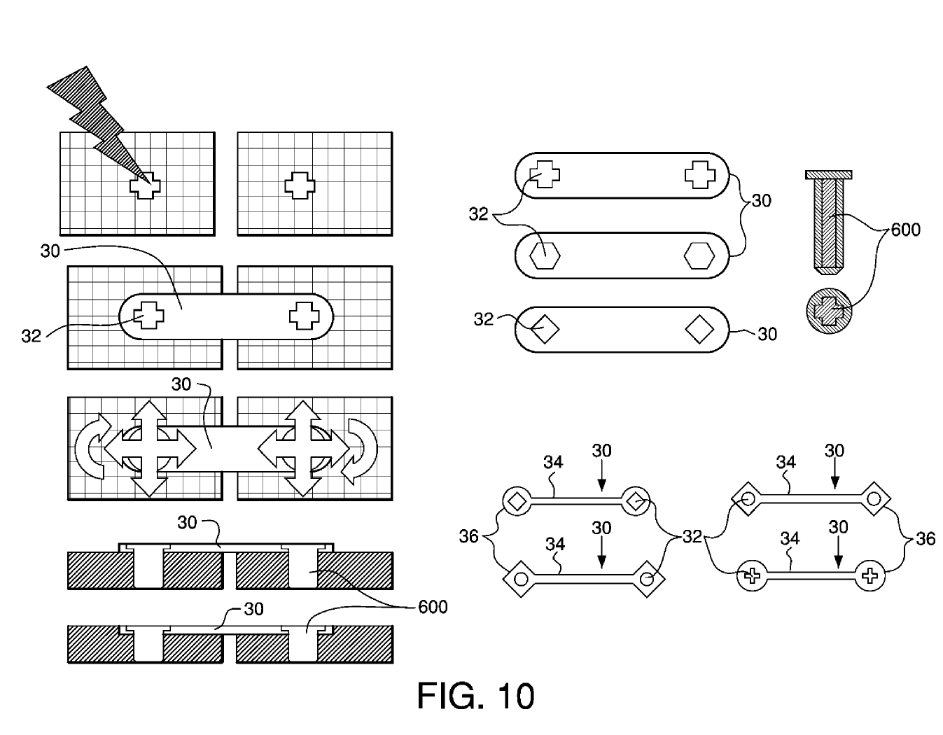

FIG. 10 illustrates additional bone plate designs. As shown, the bone plates 30 may include noncircular bore holes 32 for anchoring the implant with respect to the surrounding bone tissue. Preferably, the noncircular boreholes 32 are sized and configured to accommodate anchoring pins 600 having shafts characterized by a correspondingly non-circular cross-sectional area or alternately may house non-rounded bone pegs 310 left during the machining of the implant-receiving recess. Anchoring pins 600 having noncircular cross-sectioned shafts provided additional resistance to expulsion as compared to circular and cylindrical threaded bone anchors, as the non-rounded bone anchors are not susceptible to rotating, and thus backing out, of the surrounding bone tissue.

If a circular cross section pin, nail, screw or anchor 600 is used, it is preferred that a minimum of two such pins, nails or anchors 600 are used to avoid rotation of the bone fragment around a single pin, nail, or anchor 600.

Preferably both the bone plate 30 and the heads of the bone anchoring pins, nails, and anchors 600, are sized and configured to lie substantially flush with the top bone surface after implantation. Alternatively, the bone plate 30 may lie atop a non-machined bone surface. In a preferred embodiment, the heads of the noncircular bone pins, nails, anchors, etc. 600 are housed within the proximal portions of the boreholes through the plate 30 such that the top surfaces of the noncircular bone pins 600 lie substantially flush with the top surface of the bone plate as well as the top surface of the bone. Alternatively, the top surfaces of the noncircular bone pins, nails, anchors, etc. 600 may lie above or below the top surface of the bone plate 30. In one embodiment, the distal cross-sectional area of the noncircular bone pin, nail, anchor, etc. 600 is larger than the proximal cross-sectional area of the noncircular bone pin, nail, anchor, etc. 600 thereby providing a slight taper along the length of the shaft of the bone pin, nail, anchor, etc. 600 such that the bone pin, nail, anchor, etc. 600 may be snapped into the bone and thereby provide additional securement of the bone pin, nail, anchor, etc. 600 and bone plate 30.

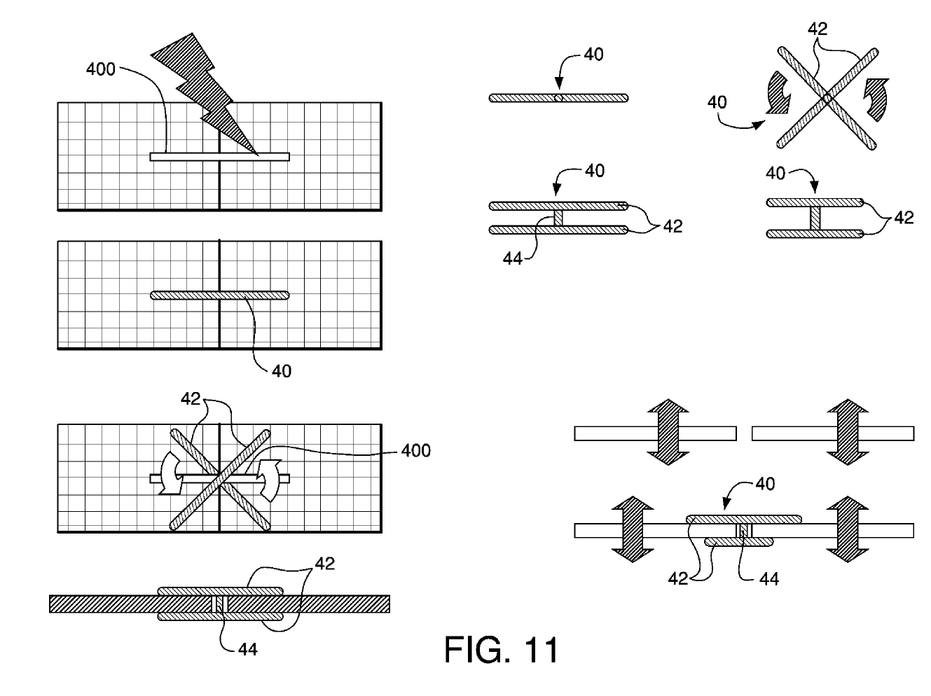

As shown in FIG. 11, the bone fixation element may be in the form of a spring-biased fixation clip 40. The spring-biased fixation clip 40 including two elongated members 42 that may be connected at or near the centers of their lengths by a connecting member 44. The connection between the two elongated members 42 biases the elongated members such that rotation of one of the elongated members 42 with respect to the other elongated member 42 is permitted. Preferably, in the absence of any external forces, the two elongated connecting members 42 are sized and configured so that they are positioned in a cruciform or “X” shape. Thereafter, upon application of a rotational force to one or both of the elongated members 42, the connecting members 42 are permitted to rotate with respect to one another so that the two elongated members 42 may become aligned in parallel form with respect to each other.

In use, one of the elongated members 42 of the spring clip may be inserted into a machined or laser formed bone slot 400 and then, immediately upon penetration into the bone, the elongated member 42 is permitted to rotate (springs) approximately 90 degrees. That is, in use, for example, the spring biased fixation clip 40, which is particularly well suited for cranio or maxillofacial applications such as, for example, cranial flap fusion or fracture reduction to lock translation of the bone pieces, may be applied across two skull bone pieces in need of fusion or reduction by resurfacing the bone(s), such as by forming a groove 400 across both bone fragments in the vicinity of the fracture site. Preferably, the groove 400 is formed all the way or completely through the bone. The spring biased fixation clip 40 may then be introduced into the groove 400 in its parallel state, such as by using a grasping instrument or inserter, such that the distal elongated member 42 is introduced below the bottom surface of the bone, while the proximal elongated member 42 is positioned above the top surface of the bone, with the connecting member 44 spanning the depth of the bone. The instrument is then released from the implant and the fixation clip automatically reverts back to its natural state in which the two elongated members 42 assume a cruciform or X shape. In returning to its cruciform state, the two elongated members 42 position themselves with respect to the machined groove 400 such that implant repulsion is prohibited. In this manner, the spring biased fixation clip 40 serves a similar function as a conventional flap-fix, the goal being to keep the bone flap on the same level as the surrounding bone.

In addition, the elongated members 42 and/or the connecting member 44 of the spring biased fixation clip 40 may include barbs or spikes or other surface texturing that assist in bone purchase and/or bone in-growth. The spring clip 40 may be loaded into a groove 400 that is one millimeter deep and five millimeters in length. The spring clip may be formed of a bioresorbable material or non-resorbable material such as stainless steel, titanium, nitinol, or PEEK.

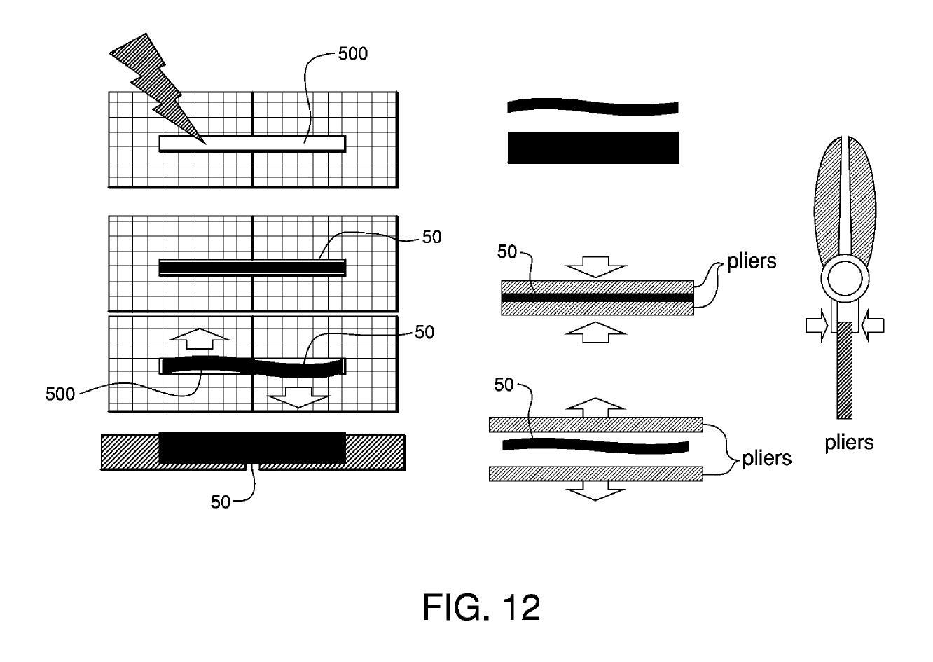

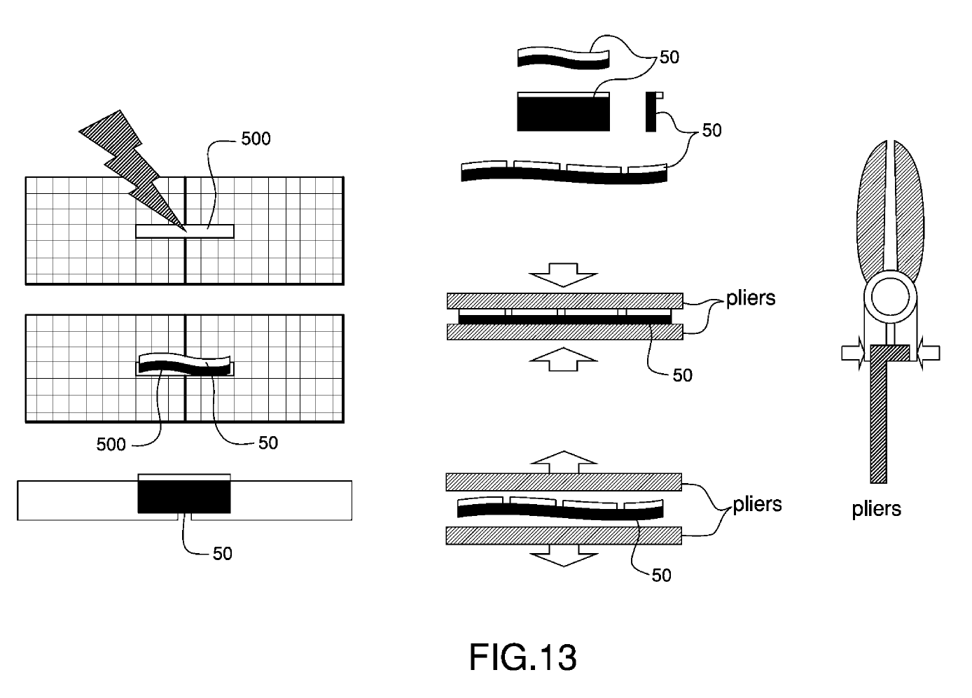

As shown in FIGS. 12 and 13, the implant may be in the form of a wave-blade implant 50 that is characterized by two states, (1) a preloaded (e.g. straight) and (2) non-loaded (e.g. wavy) configuration. In this manner, the wave-blade implant 50 may be inserted into a groove or rectilinear slot 500 formed in the bone surface in its preloaded (e.g. straight) configuration using a grasping type insertion instrument. Upon being seated in the groove 500, the wave-blade implant 50 is released from the grasping type insertion instrument and reverts back to its non-loaded (e.g. wavy) configuration. In the non-loaded configuration, the wave blade implant 50 may assume a sine-wave shape whose corners and sides contact and/or engage (e.g. dig into) the surrounding bone tissue to enhance implant seating.

The wave blade implant 50 may be formed of any biocompatible material known in the art including but not limited to cold-worked titanium, cold-worked steel, or any other flexible biocompatible material. The grasping insertion instrument may be in the form of a pliers-type instrument having a straight slot into which the wave-blade implant 50 is pre-loaded. The wave-blade implant 50 may then be pushed out of the slot of the pliers-type instrument and simultaneously inserted into the bone slot 500.

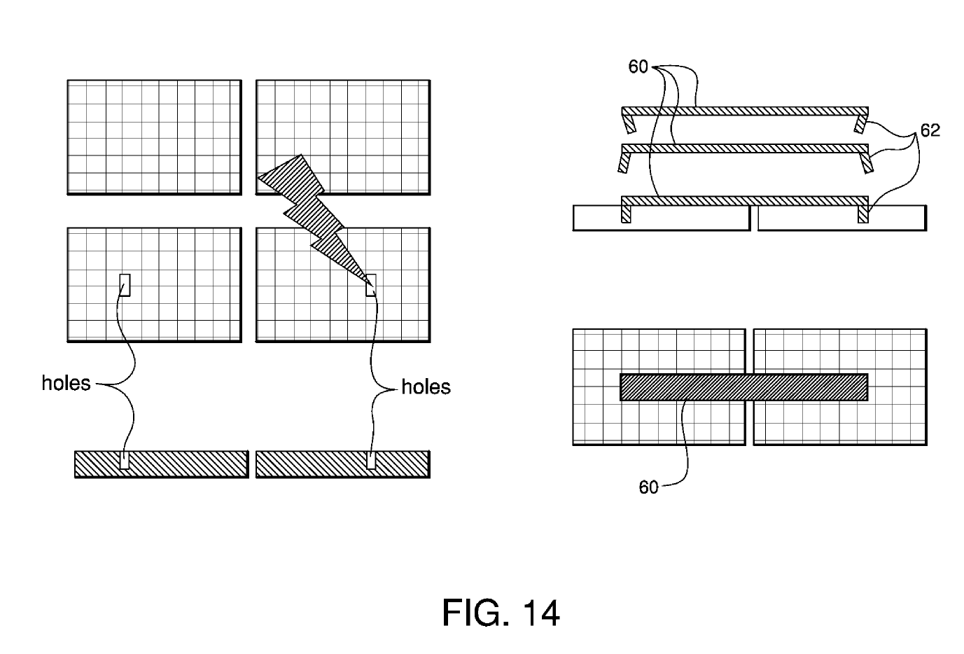

As shown in FIG. 14, the implant may be in the form of a staple-type implant 60. The staple type implant may have a plurality of legs 62, the legs 62 may be configured with a square cross-sectional area. The legs 62, preferably the square legs, of the staple type implant 60 provide angular stability and inhibit and/or prevent the bone fragments from rotating about the legs' axes. As shown, the staple type implant 60 preferably also includes diverging legs 62, the diverging direction of the legs 62 secures the staple to the bone, as they are elastically bent during the insertion.

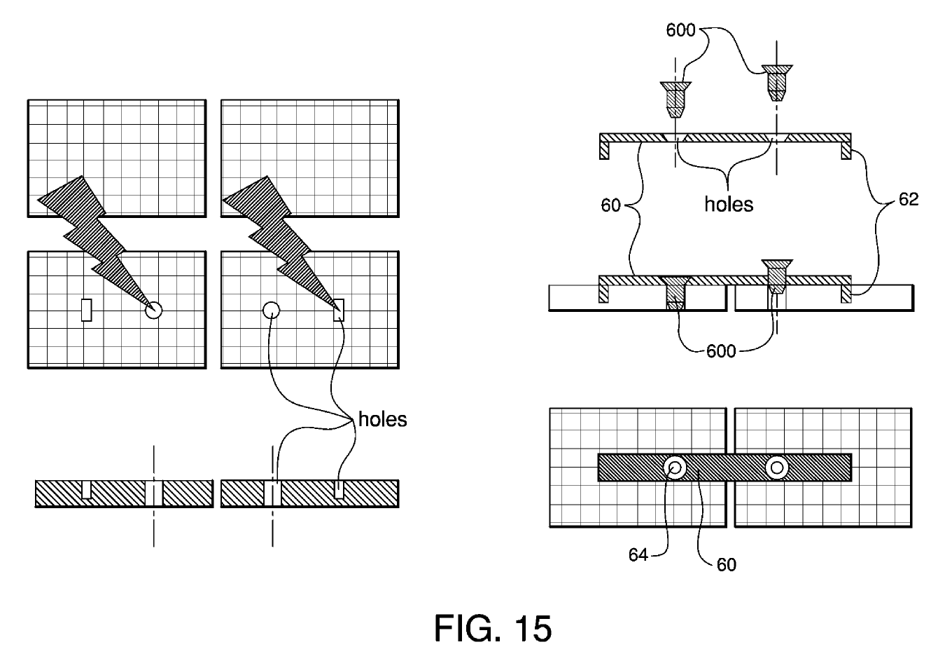

Alternatively and/or in addition, as shown in FIG. 15, the staple type implant 60 may also include one or more screw holes 64 in combination with the square cross sectional area legs 60. The screws provide additional securement to keep the staple type implant atop of the bone.

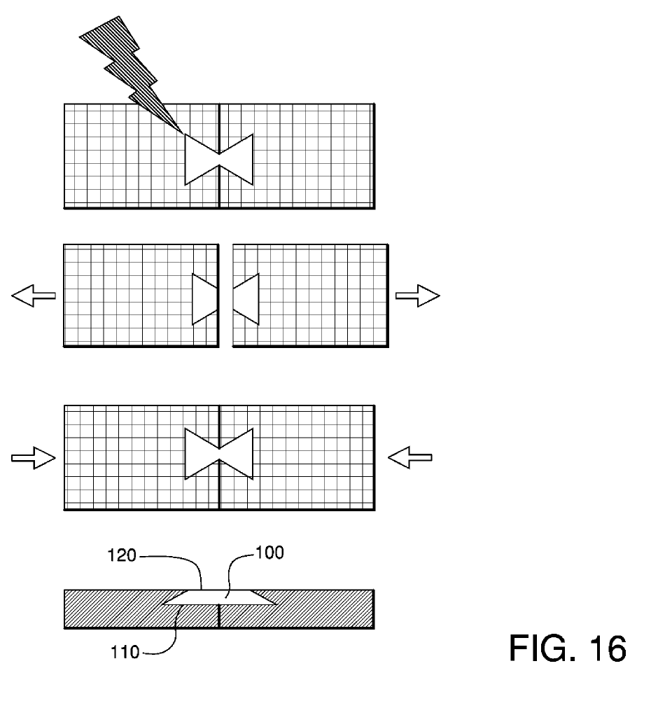

As previously stated and as best shown in FIG. 16, the implant may further include a slight taper 100. The taper may be provided along the height of the implant such that the surface area at the distal surface 110 of the implant is larger than the surface area at the proximal surface 120 of the implant. The tapered implant improves implant retention with respect to the surrounding bone tissue. The tapered height implant may be implanted by, for example, lacing the implant through one end, inserting the implant down from above with some force, snapping the implant into the receiving bed, distracting the bone segments so that the groove is slightly enlarged as may be practical in the case where there is a complete fracture, etc.

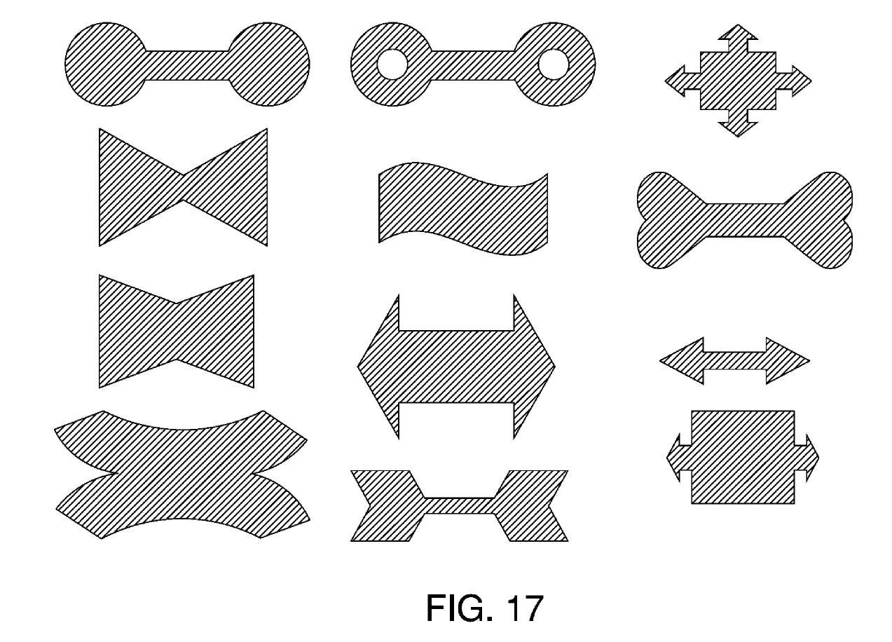

FIG. 17 illustrates a variety of alternate implant designs in accordance with another aspect of the present invention. As shown, the implants preferably include a reduced intermediary portion and enlarged end portions. The implants are preferably sized and configured to be received within corresponding receiving beds formed in the cortical bone surfaces. As such, the implants preferably have, once implanted, a zero height or reduced height profile with respect to the top bone surface as well as improved implant retention.

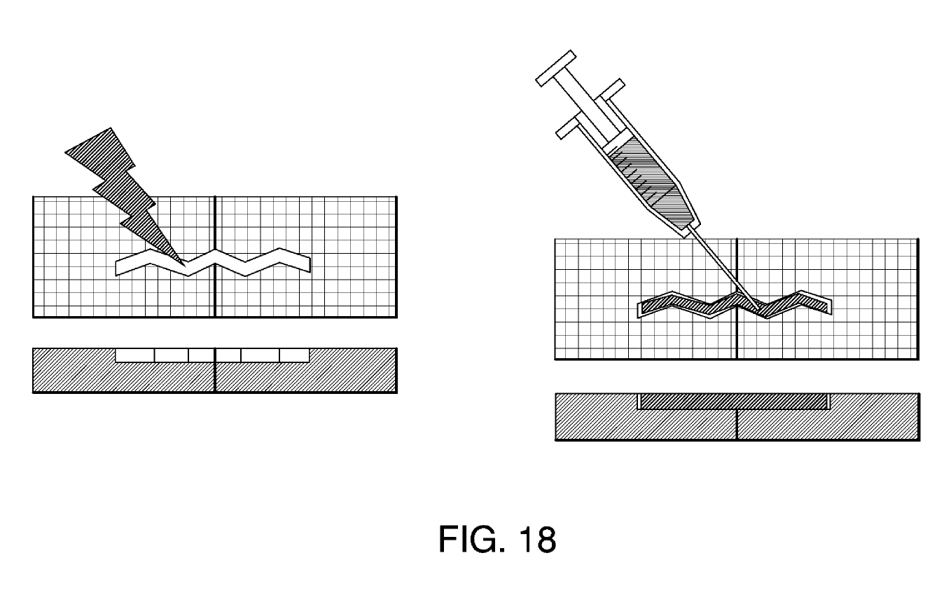

FIG. 18 illustrates a non-straight (e.g., snake-like or crooked) bone cut, which is subsequently filled by an injectable material that is hardenable in situ. The injectable material may include but is not limited to bone glue, cement, heated resorbable or non-resorbable polymer, etc. The injectable material, upon hardening, serves as a formable implant that serves the same skeletal repair purposes as the implants discussed above.

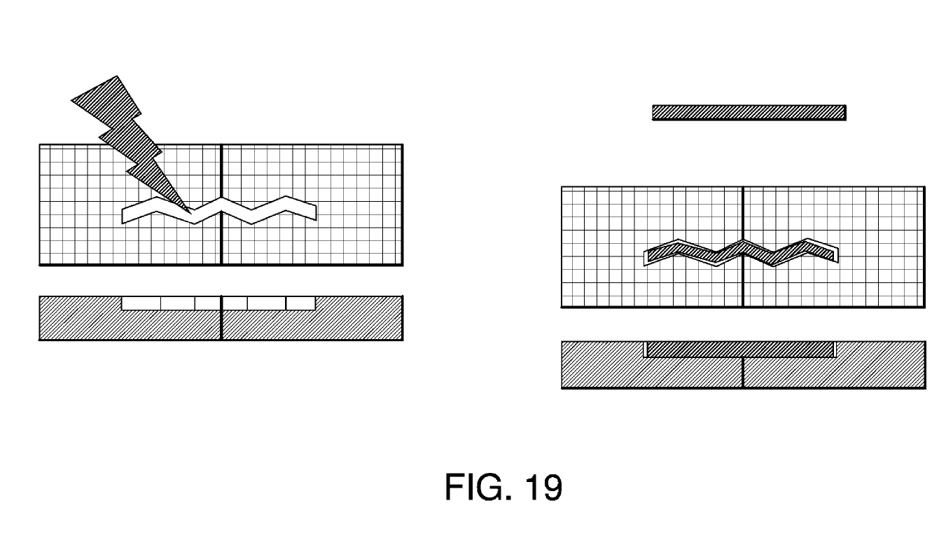

FIG. 19 illustrates a non-straight (e.g., snake-like or crooked) bone cut, which is subsequently filled with a soft and/or malleable but non-liquid material that may or may not stiffen after implantation. The stiffening material may include but is not limited to a polymeric material which is heated over the glass transition temperature.

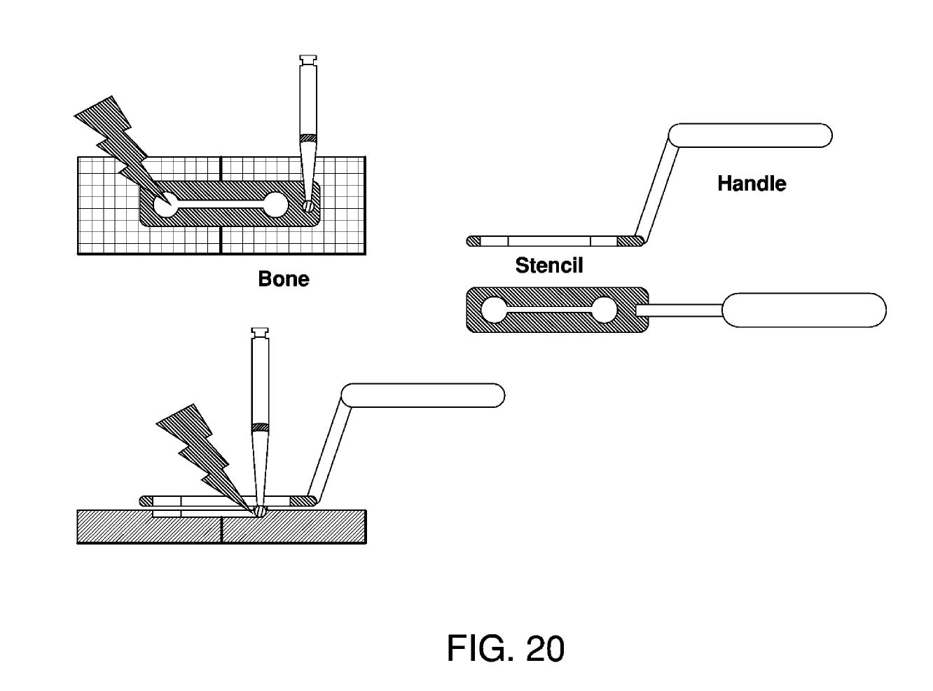

FIG. 20 illustrates a stencil or template-type instrument to guide a bone cutting tool, such as, for example, a laser, water jet, piezoelectric cutting knife, mechanical milling instrument or other bone resurfacing instrument, etc.

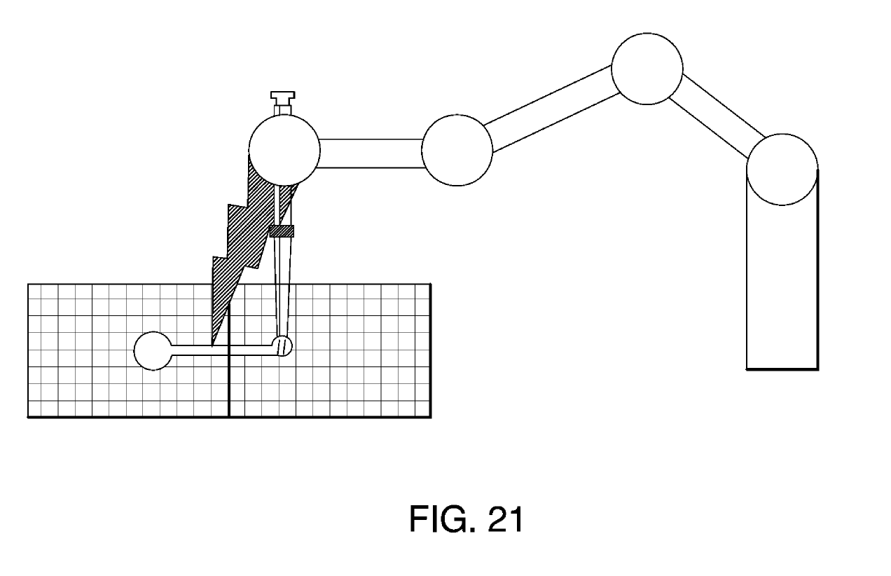

FIG. 21 illustrates the use of a numerical controlled guiding system for controlled bone removal that can assist in the precision bone resurfacing step.

It is understood that the implants provided by the present invention and methods associated therewith may utilize additional securement means, such as taking advantage of biocompatible adhesives such as cyanoacrylates, polyurethanes, epoxies, and acrylics with or without ultrasonic energy application, or may take advantage of bone-welding technology.

While the foregoing description and drawings represent the preferred embodiments of the present invention, it will be understood that various additions, modifications and substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed embodiments are therefore to be considered in all respects as illustrative and not restrictive, the scope of the invention being indicated by the appended claims, and not limited to the foregoing description.

What is claimed:

1. A bone fixation system for implanting into a recess in a bone, comprising:
a wire adapted to be implanted into the recess in the bone, the wire having a transverse cross-sectional dimension that is less than the depth of the recess in the bone; and
a biocompatible adhesive disposed in the recess and around the wire such that the wire is fixed in the recess.

2. The bone fixation system of claim 1, wherein the wire has a circular transverse cross section having a diameter of about one millimeter.

3. The bone fixation system of claim 1, wherein the wire comprises at least one extension disposed along its length.

4. The bone fixation system of claim 1, wherein the wire comprises at least one arrowhead at one end of its length.

5. The bone fixation system of claim 1, wherein the wire comprises at least one eyelet at one end of its length.

6. The bone fixation system of claim 5, wherein the recess in the bone defines at least one bone peg that is adapted to be received in the at least one eyelet.

7. The bone fixation of claim 1, wherein the wire is formed of a material that expands once introduced into a patient's body.

8. The bone fixation of claim 1, wherein the wire is drug eluting.

9. The bone fixation of claim 1, wherein the wire is coated with a tissue-ingrowth-enhancing material.

10. A bone fixation system for implanting into a recess in a bone, comprising a plate adapted to be implanted into the recess in the bone, the plate comprising two lobes at opposite ends connected by an intermediate linking portion, the plate having a thickness that is less than the depth of the recess in the bone, the intermediate linking portion having a width that is smaller than a width of the lobes.

11. The bone fixation system of claim 10, wherein each of the lobes define a bore hole extending through the plate.

12. The bone fixation system of claim 10, wherein the bore holes are adapted to receive bone anchors such that the plate may be fixed to the bone.

13. The bone fixation system of claim 12, wherein the recess in the bone defines at least one bone peg that is adapted to be received in one of the bore holes.

14. The bone fixation system of claim 10, further comprising a biocompatible adhesive disposed in the recess and around the plate such that the plate is fixed in the recess.

15. The bone fixation of claim 1, wherein the plate is formed of a material that expands once introduced into a patient's body.

16. The bone fixation of claim 1, wherein the plate is drug-eluting.

17. The bone fixation of claim 1, wherein the plate is coated with a tissue-ingrowth-enhancing material.

18. A method of fixing a fracture of a bone comprising the steps of:
forming a recess on a surface of the bone, the recess having a depth below the surface of the bone and extending across at least a portion of the fracture;
placing an implant in the recess of the bone, the implant having a shape that substantially corresponds to a shape of the recess and having a thickness that is less than the depth of the recess such that the implant is flush or is below the surface of the bone; and
fixing the implant to the bone.

19. The method of claim 15, wherein the step of fixing the implant to the bone comprises putting a biocompatible adhesive in the recess and around the implant such that the implant is fixed in the recess.

20. The method of claim 15, wherein the step of fixing the implant to the bone comprises placing a bone anchor through the implant and into the bone such that the implant is fixed in the recess.
